(19) United States
(12) Patent Application Publication
(10) Pub. No.: US 2023/0128546 A1
(43) Pub. Date: Apr. 27, 2023

(54) DATA PROCESSING APPARATUS, DATA PROCESSING METHOD AND STORAGE MEDIUM STORING PROGRAM

(71) Applicants: Takahisa OGAWA, Tokyo (JP); INFIC Inc., Shizuoka (JP)

(72) Inventor: Takahisa OGAWA, Tokyo (JP)

(21) Appl. No.: 18/048,038

(22) Filed: Oct. 20, 2022

Related U.S. Application Data
(60) Provisional application No. 63/271,101, filed on Oct. 22, 2021.

(51) Int. Cl. A61M 21/02; G06N 20/00

(52) U.S. Cl. CPC A61M 21/02; G06N 20/00; A61M 2021/0027

(57) ABSTRACT

A data processing apparatus selects content to be provided as a stimulus. The data processing apparatus includes a selecting part that selects the first content to be provided to a user as a stimulus from a plurality of pieces of content, and an acquiring part that acquires sleep data indicating sleep quality of the user provided with the first content selected by the selecting part. The selecting part selects the second content having an attribute different from that of the first content on the basis of a relationship between an attribute of a stimulus that the first content provides to the user, and sleep quality indicated by sleep data.

### CROSS-REFERENCE TO RELATED APPLICATIONS

The present application claims priority to the Provisional Patent Application number 63/271,101, filed on Oct. 22, 2021, contents of which are incorporated herein by reference in their entirety.

### BACKGROUND OF THE INVENTION

The present disclosure relates to a data processing apparatus, a data processing method, and a storage medium.

It is known that providing a stimulus such as music or images to a patient with dementia makes it possible to promote daytime waking and nighttime sleep, and produces a certain effect in improving dementia by remedying insomnia and day-night reversal (for example, see Japanese Unexamined Patent Application Publication No. 2020-161187).

In conventional therapy, it is assumed that a stimulus such as predetermined music or an image is provided to a person (hereinafter referred to as a “user”) who is to have their sleep quality improve. However, there is a case where providing a stimulus is not effective since a type of a stimulus effective for improving sleep quality depends on the user. Accordingly, it is necessary to provide a stimulus effective for improving sleep quality for each user.

### BRIEF SUMMARY OF THE INVENTION

The present disclosure focuses on this point, and its object is to enhance an effect of improving sleep quality by providing a stimulus to a user.

In the first aspect of the present disclosure, a data processing apparatus includes a selecting part that selects first content to be provided to a user as a stimulus from a plurality of pieces of content, and an acquiring part that acquires sleep data indicating sleep quality of the user provided with the first content selected by the selecting part, wherein the selecting part selects second content having an attribute different from that of the first content, on the basis of a relationship between an attribute of the first content affecting contents of a stimulus that the first content provides to the user and sleep quality indicated by the sleep data.

In the second aspect of the present disclosure, a data processing method, executed by a computer, includes the steps of selecting first content to be provided to a user as a stimulus from a plurality of pieces of content, acquiring sleep data indicating sleep quality of the user provided with the selected first content, and selecting second content having an attribute different from that of the first content on the basis of a relationship between an attribute of the first content affecting contents of a stimulus that the first content provides to the user and sleep quality indicated by the sleep data.

A storage medium according to the third aspect of the present disclosure stores, in a non-transitory manner, a program for causing a computer to execute the steps of selecting first content to be provided to a user as a stimulus from a plurality of pieces of content, acquiring sleep data indicating sleep quality of the user provided with the selected first content, and selecting second content having an attribute different from that of the first content on the basis of a relationship between an attribute of the first content affecting contents of a stimulus that the first content provides to the user and sleep quality indicated by the sleep data.

### BRIEF DESCRIPTION OF THE DRAWINGS

FIG. 1 illustrates an outline of a data processing system S.

FIG. 2 shows a configuration of a data processing apparatus 1A according to the first embodiment.

FIG. 3 shows an example of a data structure of user data.

FIG. 4 is a flowchart showing a flow of processing in the data processing apparatus 1A.

FIG. 5 shows a configuration of a data processing apparatus 1B according to a modified example.

FIG. 6 shows a configuration of a data processing apparatus 1C according to the second embodiment.

FIG. 7 is a flowchart showing a flow of processing in the data processing apparatus 1C according to the second embodiment.

### DETAILED DESCRIPTION OF THE INVENTION

Hereinafter, the present disclosure will be described through exemplary embodiments, but the following exemplary embodiments do not limit the invention according to the claims, and not all of the combinations of features described in the exemplary embodiments are necessarily essential to the solution means of the invention.

### Outline of the Data Processing Apparatus1

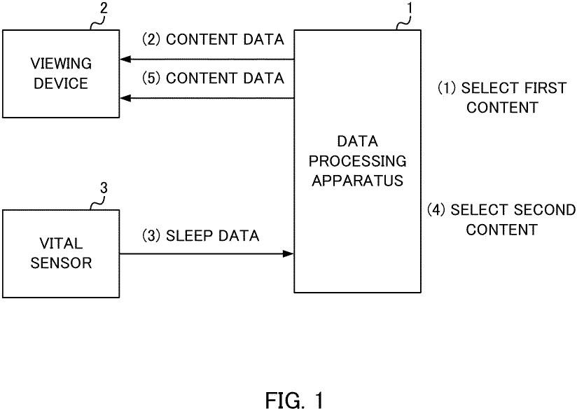

FIG. 1 illustrates an outline of a data processing system S. First, the outline of the data processing system S will be described with reference to FIG. 1. The data processing system S is a system for selecting content suitable for improving sleep quality of a patient. The data processing system S is a system that selects first content to be provided to a user as a stimulus, and further selects second content to be provided to the user as a stimulus by receiving feedback concerning sleep quality of the user provided with the selected content, in order to improve the sleep quality of the user. The data processing system S includes a data processing apparatus 1, a viewing device 2, and a vital sensor 3.

The data processing apparatus 1 is an apparatus for selecting content to be provided to the user as a stimulus. The viewing device 2 is a device for the user to view the content. The viewing device 2 is a computer, a smartphone, a tablet, a speaker, a television or the like, for example. The vital sensor 3 is a device for measuring data indicating sleep quality of the user during sleep. The vital sensor 3 is a wearable device or a smartphone capable of acquiring vital data, for example. It should be noted that content to be provided as a stimulus is music data, image data such as a movie, a video, a photograph, or art work, Augmented Reality (AR) image data, Virtual Reality (VR) image data, or the like. Hereinafter, music is taken as an example of content that the data processing apparatus 1 provides to the user as a stimulus.

The data processing apparatus 1 selects the first content to be provided to the user as a stimulus in response to a request from the user, for example. The data processing apparatus 1 transmits content data of the selected first content to the viewing device 2. The viewing device 2 plays the transmitted first content, and the user views the selected first content. If the content data transmitted by the data processing apparatus 1 is data including information for identifying the content, the viewing device 2 may acquire content corresponding to the information for identifying the content received from the data processing apparatus 1, from a storage area of the viewing device 2 or an external content server, and may play the content.

After the user has viewed the first content, the vital sensor 3 acquires sleep data indicating sleep quality of the user during sleep, and transmits the sleep data to the data processing apparatus 1. The vital sensor 3 may transmit sleep data to the data processing apparatus 1 via the viewing device 2.

The data processing apparatus 1 selects the second content to be provided to the user as a stimulus, on the basis of the information for identifying the first content provided to the user as a stimulus, and the sleep data that is acquired from the vital sensor 3 and indicates the sleep quality of the user after the first content has been provided to the user. Although details will be described later, if the sleep quality of the user after the first content has been provided to the user is poor, the data processing apparatus 1 selects the second content having a high probability of improving the sleep quality.

### First Embodiment

### Configuration of the Data Processing Apparatus 1A

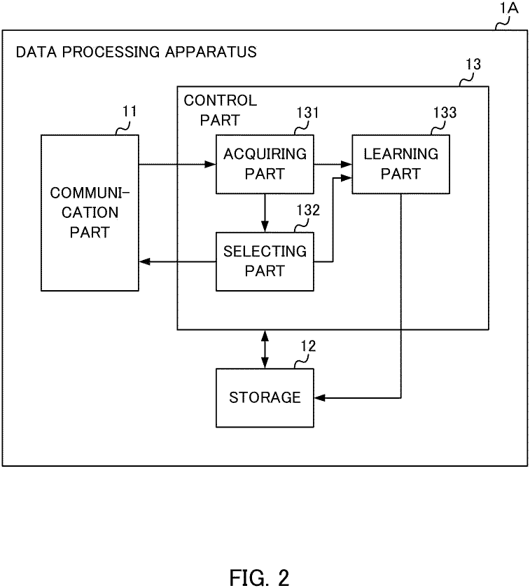

FIG. 2 shows a configuration of a data processing apparatus 1A according to the first embodiment. The data processing apparatus 1A includes a communication part 11, a storage 12, and a control part 13. The control part 13 includes an acquiring part 131, a selecting part 132, and a learning part 133.

The communication part 11 is a communication interface for communicating with the viewing device 2 and the vital sensor 3 via a network.

The storage 12 includes a storage medium, such as a Read Only Memory (ROM), Random Access Memory (RAM), or Solid State Drive (SSD). The storage 12 stores a program executed by the control part 13 in a non-transitory storage medium. The storage 12 stores user data indicating attribute data of the user. The storage 12 stores data such as a user ID, a name, a location, a date of birth, a residential location, or a hobby as the user data, for example.

The storage 12 stores a content table indicating content that can be selected by the data processing apparatus 1A. In addition to content identification information for identifying the content, attribute data indicating an attribute of the content may be stored in the content table. The attribute of the content corresponds to the nature of the content affecting contents of a stimulus provided to the user due to the content. For example, the attribute of the content is an artist who plays music included in the content, a type of an instrument used for playing the music included in the content, the frequency distribution of sound included in the content, a tempo, pitch, rhythm, harmony or melody of the music included in the content, the frequency of the user viewing the content in the past, or the probability that the user may have viewed the content in the past.

Further, the storage 12 stores user data in which a user ID, content identification information for identifying content provided to the user, a sleep date, and sleep data indicating the sleep quality of the user are associated with each other. The sleep date is the date when the content corresponding to the content identification information is provided to the user. For example, if the user is provided with the content on date D and a period of sleep lasts from date D to date D + 1 day, the sleep date is date D. The sleep date stored in the storage 12 may be a date having a certain relationship with the date on which the content corresponding to the content identification information is provided to the user, or may be a day after the date when the content is provided to the user. Since the sleep date and the date when the content is provided are equivalent, data stored in the storage 12 as the date when the content is provided may be used as data indicating the sleep date in the data processing apparatus 1.

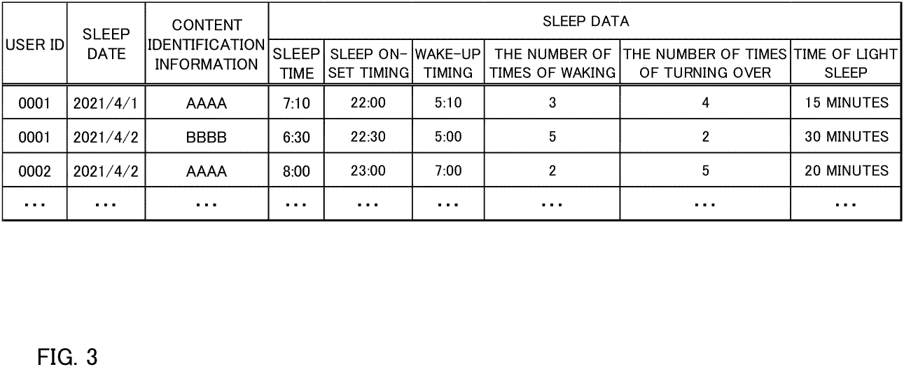

FIG. 3 shows an example of a data structure of user data. Sleep data includes at least one of sleep time, sleep-onset timing, wake-up timing, the number of times of waking, the number of times of turning over, or time of light sleep, for example. The storage 12 may store time series data of at least one of a body motion state, an amount of sweating, or heart rate during sleep acquired from the vital sensor 3, in association with a sleep date.

If a plurality of pieces of different content are provided to the user in a plurality of time periods in a day, the storage 12 may further store viewing history information indicating a relationship between a time period in which the user viewed the content and the attribute of the content. The storage 12 may store content identification information for identifying content provided to the user instead of or together with the attribute of the content, in association with a time period in which the content was provided to the user.

The control part 13 functions as an acquiring part 131, a selecting part 132, and a learning part 133 by executing a program stored in the storage 12.

The acquiring part 131 acquires sleep data indicating sleep quality of the user provided with the content selected by the selecting part 132. The acquiring part 131 acquires sleep data indicating the sleep quality of the user from the vital sensor 3 of the user via the communication part 11. The acquiring part 131 outputs the acquired sleep data to the selecting part 132, and stores the acquired sleep data in the storage 12.

The acquiring part 131 may further acquire surrounding environment data indicating a state of a surrounding environment of the user. For example, the acquiring part 131 acquires the surrounding environment data from an external server via the communication part 11. Here, the surrounding environment data is data indicating daylight hours, sunrise time, sunset time, weather, humidity, a maximum temperature, or a minimum temperature for each sleep date. The acquiring part 131 may acquire the surrounding environment data from a sensor device placed in the user’s room. The acquiring part 131 outputs the acquired surrounding environment data to the selecting part 132.

The acquiring part 131 may further acquire activity data indicating activity contents of the user. For example, the acquiring part 131 acquires schedule information including activity data indicating activity contents of the user from an external server via the communication part 11. The activity contents of the user include information indicating activities of the user in the daytime, such as gardening, reading, or yoga, for example. The acquiring part 131 outputs the acquired activity data to the selecting part 132.

The selecting part 132 selects the first content to be provided to the user as a stimulus from a plurality of pieces of content. Further, the selecting part 132 selects the second content having an attribute different from that of the first content, on the basis of a relationship between the attribute of the first content affecting contents of the stimulus provided to the user due to the first content and the sleep quality indicated by the sleep data. The second content is content that has a different attribute from that of the first content, and is capable of providing a stimulus different from that of the first content to the user, for example. The selecting part 132 selects the first content and the second content from a plurality of pieces of content stored in advance in the storage 12, for example.

By referring to a content table indicating a relationship between a title or content identification information of the content stored in the storage 12 and an attribute of the content, the selecting part 132 may identify attributes of the first content and the second content, on the basis of the titles or the content identification information of the first content and the second content, for example. Further, on the basis of a relationship between the age of the user indicated by the attribute of the user and the publication year of the content, the selecting part 132 may identify, as the attributes of the first content and the second content, a degree of the frequency with which the user viewed the content in the past or a degree of the probability that the user may have viewed the content in the past.

The selecting part 132 presents a plurality of content candidates to the user, and selects content selected by the user from the plurality of pieces of content as the first content, for example. The selecting part 132 may select a plurality of content candidates on the basis of the attribute of the user, or may select the first content on the basis of the attribute of the user. The selecting part 132 may select a plurality of content candidates or the first content further on the basis of at least one of a surrounding environment of the user indicated by the surrounding environment data or activity contents of the user indicated by the activity data.

For example, the selecting part 132 inputs the attribute of the first content and sleep data to a learned model, which is described later, and selects, as the second content, content having an attribute output from the learned model or content corresponding to content identification information output from the learned model. The selecting part 132 may select, as the second content different from the first content, content that is output from the learned model by inputting a plurality of pieces of content provided to the user before and a plurality of pieces of sleep data (e.g., sleep data shown in FIG. 3) corresponding to the dates when each piece of the content was provided, to the learned model. The sleep data corresponding to the date when the content was provided to the user is sleep data acquired during the sleep that started after the content was provided.

By referring to a table describing a correspondence relationship among the attribute of the selected first content, the sleep quality of the user provided with the first content, and the attribute of the second content to be selected, the selecting part 132 may select the second content corresponding to the attribute of the first content and the sleep quality of the user provided with the first content. For example, the selecting part 132 selects the second content having an attribute corresponding to the attribute of the selected first content and to a condition that sleep time of the user provided with the first content is less than a predetermined value.

If there is no content selected in the past, by referring to a table indicating a correspondence relationship between the attribute data of the user and the attribute of the content to be selected, the selecting part 132 may select content having an attribute corresponding to the attribute data of the user stored in the storage 12, as content to be provided to the user. The selecting part 132 may select, as content to be provided to the user, content that is output from a learned model by inputting the attribute data of the user stored in the storage 12, to the learned model created by learning, using attribute data of a learning user, an attribute of content provided to the learning user, and sleep data of the learning user provided with the content, as training data.

The selecting part 132 may use a learned model also in the selection of the second content. The selecting part 132 inputs the attribute data of the first content and the sleep data measured after the first content has been provided, to a learned model created by learning using i) learning sleep data, ii) an attribute of learning content provided to the learning user whose learning sleep data has been measured, and iii) an improvement result of sleep quality of the learning user after the learning content has been provided, as training data. The improvement result is information indicating whether or not a value of a predetermined item is improved compared to a past sleep date (e.g., the sleep date immediately before the sleep date). The selecting part 132 selects the second content corresponding to the attribute of content that is output from the learned model and has an effect of improving sleep quality after the first content has been provided.

The selecting part 132 acquires, from the storage 12, a learned model created by the learning part 133 learning i) content identification information of content provided to a plurality of learning users, ii) attribute data indicating one or more attributes of the content among an artist, genre, publication year, adjustments, tempo, pitch, rhythm, harmony, melody, or the like of the content provided to the learning user, and iii) an improvement result of sleep quality of the learning user after the learning content has been provided, as training data. The learning user is a person whose sleep quality has been measured to create the learned model.

The selecting part 132 inputs, to the learned model, content identification information of the first content and the improvement result of the sleep quality of the user provided with the first content, and selects content highly correlated with the content that is output from the learned model and has an effect for improving the sleep quality, as the second content. Highly correlated content is content in which a preset number of the attributes of the content are the same as attributes in target content, for example.

The selecting part 132 may use any approach to determine whether or not sleep quality is improved. The selecting part 132 determines whether or not the sleep quality is improved on the basis of whether or not a value of a predetermined item is improved compared to a past sleep date (e.g., the sleep date immediately before the sleep date), or whether or not an amount of change in a value of a predetermined item included in the sleep data is equal to or greater than a threshold, for example.

The selecting part 132 may select content effective for other users for whom a relationship between the content provided to the user and the sleep quality of the user provided with the content is similar. In this case, the selecting part 132 acquires, from the storage 12, a learned model created by the learning part 133 learning a relationship between content provided to each of a plurality of learning users and sleep quality of each of the plurality of users on the date when the content is provided. Then, the selecting part 132 identifies another user for whom the similarity in a relationship between the content provided to the user and an improved state of sleep quality after the content has been provided is greater than a threshold. The selecting part 132 may select content to be provided to the user as a stimulus, from one or more pieces of content effective for other users with high similarity.

The selecting part 132 may select the first content and the second content without receiving a content selection instruction from the user. The selecting part 132 acquires the content identification information of the first content and the sleep data of the user provided with the first content at a predetermined timing, without a selection operation of the content by the user. Then, the selecting part 132 selects the second content on the basis of the attribute data of the first content identified on the basis of the content identification information of the first content and the sleep data of the user provided with the first content. Since the selecting part 132 selects content without receiving the selection operation, it is possible to provide content that improves sleep quality also for a user unfamiliar with operation of devices.

The selecting part 132 selects the second content if the sleep quality indicated by the second sleep data, which is sleep data in the second sleep period including one or more sleep dates after the first content has been provided to the user as a stimulus, is not improved compared to sleep quality indicated by the first sleep data, which is sleep data in the first sleep period including one or more sleep dates before the first content is provided to the user as a stimulus. The selecting part 132 compares the first sleep data that is sleep data of the user in the first sleep period, which is a predetermined period before the first content is provided, with the second sleep data that is sleep data of the user in the second sleep period, which is a predetermined period during which the first content is provided, to determine whether or not the sleep quality is improved. The selecting part 132 selects the second content if the second sleep data does not indicate improved sleep quality compared with the first sleep data. The predetermined period is one day, three days, or one week, for example. The lengths of the first sleep period and the second sleep period may be different.

The selecting part 132 selects the second content if the sleep quality indicated by the sleep data after the first content has been provided to the user as a stimulus is poorer than a predetermined threshold. The selecting part 132 selects the second content if a predetermined item among items included in the sleep data of the user over a predetermined period during which the first content is provided is equal to or less than a predetermined threshold. The predetermined threshold is a value obtained by adding a predetermined value to a target value set for each user or a value of an item included in sleep data of the user before the content is provided, for example. In a case where average sleep time of the user is set to be equal to or less than 4 hours as a threshold, if the average sleep time of the user in a predetermined period included in the first sleep data is equal to or less than 4 hours, the selecting part 132 may select the second content, for example.

The selecting part 132 may select the first content and the second content further on the basis of attribute data indicating an attribute of the user. For example, the selecting part 132 refers to data in which the attribute of the user, such as a location, a residential location, a date of birth or age, or a hobby of each of a plurality of learning users, and content suitable for improving sleep quality of a user having each attribute are associated with each other, thereby selecting the first content and the second content suitable for the attribute of the user. The selecting part 132 selects content in which the user’s hometown is included in lyrics, content that tends to be preferred by a person of the user’s generation, or content related to the user’s hobby, for example. The selecting part 132 selecting such content satisfies the user, allowing the user to get good sleep.

On the basis of a relationship between the attribute of the first content selected on the basis of the attribute of the user and the sleep data indicating the sleep quality of the user provided with the first content, the selecting part 132 may select the second content that is suitable for the attribute of the user and may improve sleep quality. As an example, if the sleep quality after the first content, selected on the basis of the first attribute (e.g., age) among the attributes of the user, that has been provided to the user is equal to or greater than an allowable level, the selecting part 132 selects the second content that has the same first attribute as the first content and is different from the first content. The selecting part 132 selecting such second content makes it possible to maintain good sleep quality without having the user repeatedly view the same content.

On the other hand, if sleep quality after the first content, selected on the basis of the first attribute (e.g., age) among the attributes of the user, that has been provided to the user is less than an allowable level, the selecting part 132 selects the second content on the basis of the second attribute (e.g., location) that is different from the first attribute. The selecting part 132 operating in this manner increases the probability of being able to select the second content that can improve the sleep quality of the user.

The selecting part 132 may select the second content on the basis of the sleep quality indicated by the sleep data measured after the user provided with the first content fell asleep, and the attribute of this user, using a learned model created by learning using a relationship among i) the attribute data, ii) a plurality of pieces of content provided to each of a plurality of learning users, and iii) an improvement result of sleep quality of each of a plurality of learning users on the date when the content was provided, as training data. In this case, the selecting part 132 inputs the attribute data of the user, the first content provided to the user, and the sleep data of the user measured after the first content has been provided, to this learned model stored in the storage 12. The selecting part 132 selects content output from the learned model as the second content to be provided to the user as a stimulus.

Incidentally, sleep quality may be further improved by optimizing the time at which to provide the user with content. The selecting part 132 may select a plurality of pieces of second content having different attributes corresponding to a plurality of time periods. Specifically, the selecting part 132 selects the plurality of pieces of second content such that a combination of the attributes of the plurality of pieces of second content to be provided to the user in each of the plurality of time periods is to be a combination suitable for improving sleep quality. For example, the selecting part 132 selects the second content with a fast tempo in a daytime time period, and selects the second content with a slow tempo in a nighttime time period. The plurality of time periods may be a time period corresponding to the user’s activity, such as a time of waking up, after breakfast, after lunch, after dinner, or before going to bed, or may be a time period determined by timings, for example.

The selecting part 132 may select the second content to be provided to the user in each of the plurality of time periods. For example, the selecting part 132 selects the second content with a relatively fast tempo in a daytime time period for a user who tends to sleep in the daytime, and selects the second content with a relatively slow tempo in a daytime time period for a user who tends to be restless in the daytime. Since the selecting part 132 configured in this manner can provide the second content having an attribute suitable for the attribute of the user in each time period, sleep quality can be easily improved.

The selecting part 132 may select the plurality of pieces of second content corresponding to a plurality of time periods, using a learned model. In this case, the selecting part 132 acquires, from the storage 12, a learned model created by the learning part 133 learning i) the attribute data of the content provided to the learning user, ii) the sleep data of the learning user, iii) information indicating the time period in which the content is provided to the learning user, and iv) the improvement result of the sleep quality of the learning user after learning content has been provided, as training data. The selecting part 132 inputs the attribute data of the content provided to the user and the sleep data of the user provided with the content, to the learned model. The selecting part 132 acquires information, output from the learned model, about a plurality of time periods in which to provide content effective for improving the sleep quality. The selecting part 132 selects one or more pieces of second content to be provided to the user in one or more of the time periods indicated by the acquired information.

By referring to data indicating influence of the surrounding environment of the user on the user’s mind and body, and data indicating contents of a stimulus suitable for the physical and mental state of the user, the selecting part 132 may select the second content to be provided to the user in the surrounding environment indicated by the surrounding environment data. The selecting part 132 refers to a table in which a relationship between data indicating the influence of the surrounding environment of the user on the user’s mind and body stored in the storage 12 and data indicating the contents of the stimulus suitable for the physical and mental state of the user is stored, and identifies the contents of the stimulus suitable for the physical and mental state of the user corresponding to the surrounding environment data acquired from the acquiring part 131. The selecting part 132 selects content corresponding to the contents of the stimulus suitable for the physical and mental state of the user as the second content.

As an example, if the surrounding environment data indicates that the temperature is high, it is assumed that the user is subject to fatigue, and therefore “music with a slow tempo” is determined as a stimulus suitable for the physical and mental state of the user. The selecting part 132 selects content corresponding to “music with a slow tempo”. Further, if the surrounding environment data indicates that the weather is rainy, it is assumed that the user is subject to a depressed feeling, and therefore “music with a fast tempo” is determined as a stimulus suitable for the physical and mental state of the user. The selecting part 132 selects content corresponding to “music with a fast tempo”.

The selecting part 132 may select content suitable for the surrounding environment using a learned model. In this case, the selecting part 132 acquires, from the storage 12, a learned model created by the learning part 133 learning i) attribute data of the learning content provided to each of a plurality of learning users, ii) the surrounding environment on the date when the plurality of learning users are provided with the learning content, and iii) the improvement result of the sleep quality of each of the plurality of users on the date when the content is provided, as the training data. Then, the selecting part 132 inputs the attribute data of the first content provided to the user, the sleep data of the user each measured on the date when the first content is provided, and the surrounding environment data to the learned model. The selecting part 132 selects content output from the learned model as the second content to be provided to the user as a stimulus in the surrounding environment of the user.

By referring to data indicating influence of activity contents of the user on the user’s body and mind, and data indicating the contents of a stimulus suitable for the physical and mental state of the user, the selecting part 132 may select the second content to be provided to a user who performed an activity indicated by the activity data. The selecting part 132 refers to a table, stored in the storage 12, in which a relationship between data indicating the influence of the activity contents of the user on the user’s body and mind and the data indicating the contents of the stimulus suitable for the physical and mental state of the user is stored, and acquires the data indicating the contents of the stimulus suitable for the physical and mental state of the user who performed the activity indicated by the activity data acquired from the acquiring part 131. The selecting part 132 selects the content corresponding to the data indicating the contents of the stimulus suitable for the physical and mental state of the user as the second content.

As an example, if the activity data indicates that the user performed a hard workout, since it is assumed that the user is subject to fatigue, the selecting part 132 determines that the contents of the stimulus suitable for the physical and mental state of the user are “music with a slow tempo”. The selecting part 132 selects content corresponding to “music with a slow tempo”. Further, if the activity data indicates that the user rarely goes out, since it is assumed that the user is subject to a depressed feeling, the selecting part 132 determines that the contents of the stimulus suitable for the physical and mental state of the user are “music with a fast tempo”. The selecting part 132 selects content corresponding to “music with a fast tempo”.

The selecting part 132 may select content suitable for the activity contents of the user using a learned model. In this case, the selecting part 132 acquires, from the storage 12, a learned model created by the learning part 133 learning i) the attribute data of the first content provided to each of the plurality of learning users and the second content, ii) the contents of the activity performed before the plurality of learning users are provided with the second content, and iii) the improvement result of sleep quality of each of the plurality of users on the date when the second content was provided after the first content, as training data. Then, the selecting part 132 inputs, to the learned model, the attribute data of the first content provided to the user, the sleep data of the user each measured on the date when the first content is provided, and the activity contents data indicating the activity contents of the user performed by the user before the second content is provided. The selecting part 132 selects the content output from the learned model as the second content to be provided to the user who performed the activity indicated by the activity contents.

The learning part 133 creates the above-described various learned models, including the learned model that learned a relationship between the content provided to the learning user and the sleep quality of the learning user provided with the content, and stores the created learned models in the storage 12.

### Flow of Processing in the Data Processing Apparatus 1A

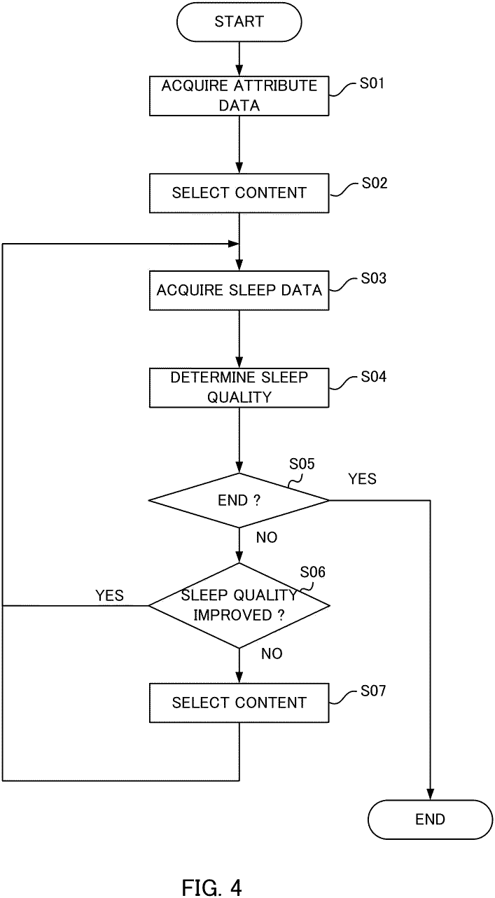

FIG. 4 is a flowchart showing the flow of processing in the data processing apparatus 1A. The processing in this flowchart starts at the time when registration processing of the user is completed and content can be selected, for example.

The selecting part 132 acquires attribute data of the user stored in the storage 12 (S01). Next, the selecting part 132 inputs the attribute data of the user to a learned model acquired from the storage 12, and selects output content as the first content to be provided to the user as a stimulus (S02).

The acquiring part 131 acquires, from the vital sensor 3, sleep data of the user provided with the first content as a stimulus (S03). The selecting part 132 determines whether or not sleep quality indicated by the acquired sleep data is improved (S04). Next, the selecting part 132 determines whether or not an ending condition is satisfied (S05). The ending condition is the case that the user has reached a desired sleep quality, for example. If the ending condition is satisfied (YES in S05), the data processing apparatus 1 ends the content selection processing. If the ending condition is not satisfied (NO in S05), the data processing apparatus 1 proceeds to S06.

If the sleep quality indicated by the sleep data is improved (YES in S06), the data processing apparatus 1 proceeds to S03. If the sleep quality indicated by the sleep data is not improved (NO in S06), the selecting part 132 inputs information for identifying the first content and the sleep quality of the user provided with the first content, to the learned model acquired from the storage 12, and selects the content output from the learned model as the second content (S07). Hereinafter, the data processing apparatus 1 provides the user with the selected second content as the first content, and repeats the content selection processing until the ending condition is satisfied.

### Effects of the First Embodiment

As described above, in the data processing apparatus 1A, the selecting part 132 selects the first content to be provided to the user as a stimulus from a plurality of pieces of content. Next, the acquiring part 131 acquires sleep data indicating the sleep quality of the user provided with the first content selected by the selecting part 132. Then, the selecting part 132 selects the second content having an attribute different from that of the first content on the basis of a relationship between the attribute of the stimulus that the first content provides to the user and the sleep quality indicated by the sleep data. The data processing apparatus 1A configured in this manner can select content having a therapeutic effect on the user.

### Modified Example

Even if improvement in the sleep quality is confirmed, the sleep quality may have been improved due to a factor other than content, such as due to a workout or the weather. Accordingly, the data processing apparatus 1 may calculate a sleep score indicating the sleep quality, evaluate influence of a factor other than the content, and correct the sleep score on the basis of the influence of the factor other than the content.

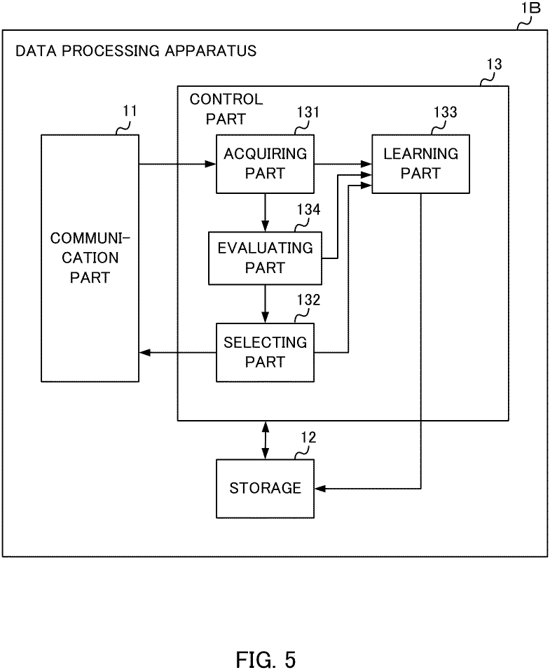

FIG. 5 shows a configuration of a data processing apparatus 1B according to a modified example. The same reference numerals are given to the same functional parts as those already described in FIG. 5, and descriptions thereof will be omitted. The data processing apparatus 1B further includes an evaluating part 134. The evaluating part 134 generates an evaluation result indicating a degree of effect due to the first content being provided to the user, on the basis of a relationship between sleep quality indicated by the first sleep data and sleep quality indicated by the second sleep data. Here, the first sleep data is sleep data of the user in the first sleep period, which is a predetermined period before the first content is provided. The second sleep data is sleep data of the user in the second sleep period, which is a predetermined period in which the first content is provided.

The evaluating part 134 calculates a sleep score indicating the sleep quality on the basis of sleep-onset timing, wake-up timing, sleep time, or an amount of movement or heart rate during sleep, for example. Then, the evaluating part 134 subtracts the second sleep score, calculated from the second sleep data, from the sleep score calculated from the first sleep data, thereby calculating the evaluation result indicating a degree of effect of the content on the improvement of the sleep data. The evaluating part 134 may calculate a difference ratio obtained by dividing the difference between the first sleep score and the second sleep score by the first sleep score, as an evaluation result. Further, the evaluating part 134 may use an average value of the sleep data acquired in a predetermined period as the first sleep data and the second sleep data. The predetermined period is three days or one week, for example.

The evaluating part 134 may correct the evaluation result using the surrounding environment data. For example, the evaluating part 134 refers to data indicating a relationship between the surrounding environment and the sleep quality, thereby identifying a degree of influence of the surrounding environment indicated by the surrounding environment data on the sleep quality. On the basis of the identified degree of influence, the evaluating part 134 generates an evaluation result by correcting a degree of effects identified on the basis of the relationship between the sleep quality indicated by the first sleep data and the sleep quality indicated by the second sleep data.

The evaluating part 134 refers to a table in which a relationship between the surrounding environment data acquired by the acquiring part 131 and a correction value of the evaluation result is described, and acquires the correction value of the evaluation result corresponding to the surrounding environment data, input from the acquiring part 131, on a target day when the sleep score is to be calculated. The evaluating part 134 corrects the first sleep score and the second sleep score by adding the acquired correction value to the calculated sleep score.

The evaluating part 134 may correct the evaluation result on the basis of the contents of the activity of the user during the daytime. By referring to the data indicating a relationship between the activity contents and the sleep quality, the evaluating part 134 identifies a degree of influence of the activity contents indicated by the activity data on the sleep quality. On the basis of the identified degree of influence, the evaluating part 134 generates an evaluation result by correcting a degree of effect identified on the basis of the relationship between the sleep quality indicated by the first sleep data and the sleep quality indicated by the second sleep data.

The evaluating part 134 acquires information indicating the activity contents of the user included in schedule information acquired by the acquiring part 131. The evaluating part 134 refers to a table in which a relationship between the activity contents of the user and the correction value of the evaluation result is described, and acquires a correction value corresponding to the activity contents of the user on the target day when the sleep score is to be calculated. The evaluating part 134 corrects the first sleep score and the second sleep score by adding the acquired correction value to the sleep score.

In the data processing apparatus 1B, the selecting part 132 calculates a degree of improvement in sleep quality using the corrected sleep score acquired from the evaluating part 134. As an example, the selecting part 132 calculates a value obtained by subtracting the corrected second sleep score from the corrected first sleep score, as the degree of improvement in sleep quality.

In addition to the sleep data of the learning user acquired by the acquiring part 131 and the learning content selected by the selecting part 132, the learning part 133 creates a learned model that learned the corrected sleep score of the learning user evaluated by the evaluating part 134 as training data, and stores the learned model in the storage 12.

Since the data processing apparatus 1B including the evaluating part 134 corrects the influence of a factor other than the content on the sleep score, and determines whether or not the content contributed to the improvement of the sleep quality with higher accuracy, the data processing apparatus 1B makes it possible to improve the accuracy of content selection.

### Second Embodiment

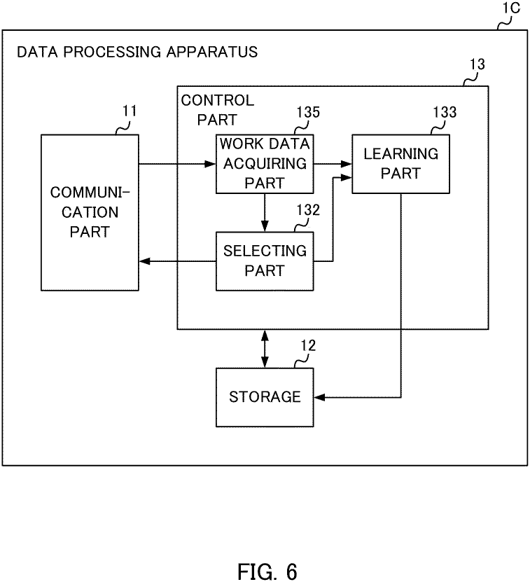

In the first embodiment, sleep quality of the user is measured using sleep data, but the sleep quality of the user may be determined on the basis of data indicating the workload of a caregiver. FIG. 6 shows an apparatus configuration of a data processing apparatus 1C according to the second embodiment. In FIG. 6, functional parts equivalent to those described above are denoted by the same reference numerals, and description thereof will be omitted.

The data processing apparatus 1C includes a work data acquiring part 135. The work data acquiring part 135 acquires work data indicating work contents of a caregiver who cares for a user provided with the first content selected by the selecting part 132. The work data acquiring part 135 acquires work data of the caregiver who cares for the user. The work data is daily report information that records work contents of the caregiver, for example.

The selecting part 132 estimates sleep quality of the user on the basis of the work contents of the caregiver indicated by the work data, and selects the second content having an attribute different from that of the first content on the basis of a relationship between the attribute of a stimulus that the first content provides to the user and the estimated sleep quality. If the work data is text data, the selecting part 132 extracts information indicating the load of the caregiver described in a daily report by performing natural language processing on work recording data. The information indicating the load of the caregiver is information indicating symptoms of a patient associated with dementia, for example. The information indicating the load of the caregiver is information indicating the presence or absence of violence against the caregiver or the like, information indicating that the user yelled or acted violently, information indicating that the user hallucinated, or information indicating the number of times that the caregiver was called for nighttime excretion or the number of times assisting with excretion, for example.

The selecting part 132 estimates the sleep quality of the user provided with the first content by referring to the data, stored in the storage 12, indicating a relationship between work contents of the caregiver and the sleep quality of the user (the care receiver), for example. The selecting part 132 may estimate the sleep quality of the user by inputting information indicating the load of the caregiver to a learned model created by learning information indicating the load of the caregiver for the learning user and the sleep quality of the learning user as training data.

The selecting part 132 selects the second content suitable for the user who needs the work contents indicated by the work data to be performed by the caregiver, after the first content has been provided using the above-described method. The selecting part 132 acquires, from the storage 12, a learned model created by the learning part 133 learning i) the first content and the second content respectively provided to each of a plurality of learning users, ii) the work data indicating the work contents of the caregiver after the plurality of learning users have been provided with the first content, and iii) an improvement result of sleep quality of each of the plurality of users on the date when the second content is provided after the first content, as training data. Then, the selecting part 132 inputs the attribute data of the first content provided to the user and the work data indicating the work contents of the caregiver after the first content has been provided to the user, to the learned model. The selecting part 132 selects the content output from the learned model as the second content.

### Flow of Processing in the Data Processing Apparatus 1C

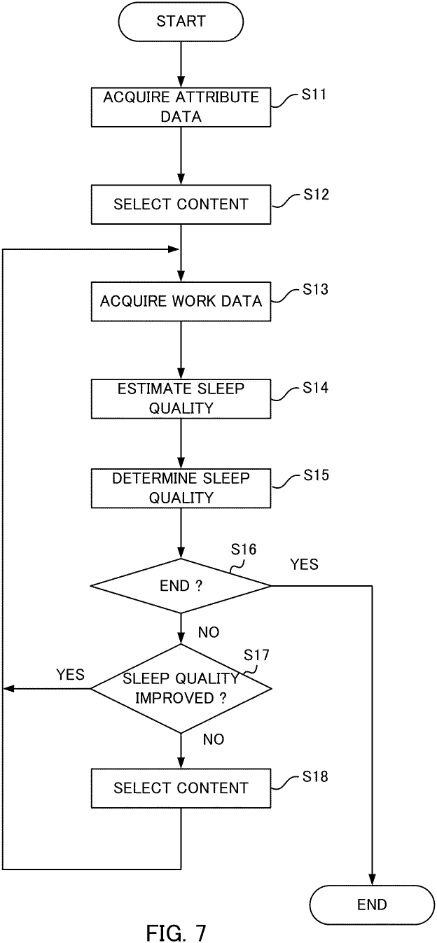

FIG. 7 is a flowchart showing the flow of processing in the data processing apparatus 1C. The processing in this flowchart starts at the time when registration processing of the user is completed and content can be selected, for example.

The selecting part 132 acquires attribute data of the user stored in the storage 12 (S11). Next, the selecting part 132 inputs the attribute data of the user to a learned model acquired from the storage 12, and selects output content as the first content to be provided to the user as a stimulus (S12).

The acquiring part 131 acquires work data of a caregiver who cares for the user (S13). The selecting part 132 extracts information indicating the work load of the caregiver from the acquired work data, and estimates sleep quality of the user on the basis of the information indicating the work load. The selecting part 132 determines whether or not the estimated sleep quality is improved (S15).

Next, the selecting part 132 determines whether or not an ending condition is satisfied (S16). The ending condition is the case that the user has reached a desired sleep quality, for example. If the ending condition is satisfied (YES in S16), the data processing apparatus 1C ends the content selection processing. If the ending condition is not satisfied (NO in S15), the data processing apparatus 1C proceeds to S17.

If the sleep quality indicated by the sleep data is improved (YES in S17), the data processing apparatus 1C proceeds to S13. If the sleep quality indicated by the sleep data is not improved (NO in S17), the data processing apparatus 1C inputs information for identifying the first content and the sleep quality of the user provided with the first content to the learned model acquired from the storage 12, and selects the output content as the second content (S18). Hereinafter, the data processing apparatus 1C provides the user with the selected second content as the first content, and repeats the content selection processing until the ending condition is satisfied.

### Effect of the Data Processing Apparatus 1C

As described above, in the data processing apparatus 1C, the selecting part 132 selects the first content to be provided to the user as a stimulus from a plurality of pieces content. Next, the work data acquiring part 135 acquires work data indicating the work contents of the caregiver who cares for the user provided with the first content selected by the selecting part 132. Then, the selecting part 132 estimates the sleep quality of the user on the basis of the work contents indicated by the work data, and selects the second content having an attribute different from that of the first content on the basis of a relationship between the attribute of the first content affecting the contents of the stimulus provided to the user and the estimated sleep quality. The data processing apparatus 1C configured in this manner can select content having a therapeutic effect also for a user from which sleep data cannot be acquired.

The present disclosure is explained on the basis of the exemplary embodiments. The technical scope of the present disclosure is not limited to the scope explained in the above embodiments and it is possible to make various changes and modifications within the scope of the disclosure. For example, the specific embodiments of the distribution and integration of the apparatus are not limited to the above embodiments, all or part thereof, can be configured with any unit which is functionally or physically dispersed or integrated. Further, new exemplary embodiments generated by arbitrary combinations of them are included in the exemplary embodiments. Further, effects of the new exemplary embodiments brought by the combinations also have the effects of the original exemplary embodiments.

What is claimed is:

1. A data processing apparatus comprising:
a selecting part that selects first content to be provided to a user as a stimulus from a plurality of pieces of content, and
an acquiring part that acquires sleep data indicating sleep quality of the user provided with the first content selected by the selecting part, wherein
the selecting part selects second content having an attribute different from that of the first content, on the basis of a relationship between an attribute of the first content affecting contents of a stimulus that the first content provides to the user and sleep quality indicated by the sleep data.

2. The data processing apparatus according to claim 1, wherein
the acquiring part further acquires attribute data indicating an attribute of the user, and
the selecting part selects the first content and the second content further on the basis of the attribute data.

3. The data processing apparatus according to claim 1, wherein
the data processing apparatus further includes a storage that stores the sleep data in association with a sleep date, wherein
the selecting part selects the second content if sleep quality indicated by second sleep data, which is sleep data in a second sleep period including one or more sleep dates after the first content has been provided to the user as a stimulus, is not improved compared to sleep quality indicated by first sleep data, which is sleep data in a first sleep period including one or more sleep dates before the first content is provided to the user as a stimulus.

4. The data processing apparatus according to claim 3, wherein
the data processing apparatus further includes an evaluating part that generates an evaluation result indicating a degree of an effect due to the first content being provided to the user, on the basis of a relationship between the sleep quality indicated by the first sleep data and the sleep quality indicated by the second sleep data.

5. The data processing apparatus according to claim 4, wherein
the acquiring part further acquires surrounding environment data indicating a state of a surrounding environment of the user, and
the evaluating part identifies a degree of influence of the surrounding environment indicated by the surrounding environment data on sleep quality by referring to data indicating a relationship between the surrounding environment and the sleep quality, and generates the evaluation result by correcting the degree of effect identified on the basis of the relationship between the sleep quality indicated by the first sleep data and the sleep quality indicated by the second sleep data, on the basis of the identified degree of influence.

6. The data processing apparatus according to claim 4, wherein
the acquiring part further acquires activity data indicating activity contents of the user, and
the evaluating part identifies a degree of influence of the activity contents indicated by the activity data on sleep quality by referring to data indicating a relationship between the activity contents and the sleep quality, and generates the evaluation result by correcting the degree of effect identified on the basis of the relationship between the sleep quality indicated by the first sleep data and the sleep quality indicated by the second sleep data, on the basis of the identified degree of influence.

7. The data processing apparatus according to claim 1, wherein
the selecting part selects the second content if the sleep quality indicated by the sleep data after the first content has been provided to the user as a stimulus is poorer than a predetermined threshold.

8. The data processing apparatus according to claim 1, wherein
the selecting part inputs attribute data of the first content and the sleep data measured after the first content has been provided, to a learned model created by learning using learning sleep data, an attribute of learning content provided to a learning user whose learning sleep data has been measured, and an improvement result of sleep quality of the learning user after the learning content has been provided, as training data, to select the second content corresponding to an attribute output by the learned model.

9. The data processing apparatus according to claim 1, wherein
the selecting part inputs attribute data of the first content, attribute data of the user, and the sleep data measured after the first content has been provided, to a learned model created by learning using learning sleep data, an attribute of learning content provided to a learning user whose learning sleep data has been measured, an attribute of the learning user, and an improvement result of sleep quality of the learning user after the learning content has been provided, as training data, to select the second content corresponding to an attribute output by the learned model.

10. The data processing apparatus according to claim 1, wherein
the selecting part selects the first content and the second content without receiving a content selection instruction from the user.

11. The data processing apparatus according to claim 1, wherein
the selecting part selects a plurality of pieces of the second content each having a different attribute corresponding to a plurality of time periods.

12. The data processing apparatus according to claim 1, wherein
the acquiring part further acquires surrounding environment data indicating a state of a surrounding environment of the user, and
the selecting part refers to data indicating an influence of the surrounding environment of the user on the user’s body and mind, and data indicating contents of a stimulus suitable for a physical and mental state of the user, to select the second content to be provided to the user in the surrounding environment indicated by the surrounding environment data.

13. The data processing apparatus according to claim 1, wherein
the acquiring part further acquires activity data indicating activity contents of the user, and
the selecting part refers to data indicating an influence of the activity contents of the user on the user’s body and mind, and data indicating contents of a stimulus suitable for a physical and mental state of the user, to select the second content to be provided to the user who did the activity indicated by the activity data.

14. A data processing method, executed by a computer, comprising the steps of:
selecting first content to be provided to a user as a stimulus from a plurality of pieces of content;
acquiring sleep data indicating sleep quality of the user provided with the selected first content; and
selecting second content having an attribute different from that of the first content on the basis of a relationship between an attribute of the first content affecting contents of a stimulus that the first content provides to the user and sleep quality indicated by the sleep data.

15. A non-transitory storage medium storing a program for causing a computer to execute the steps of:
selecting first content to be provided to a user as a stimulus from a plurality of pieces of content;
acquiring sleep data indicating sleep quality of the user provided with the selected first content; and
selecting second content having an attribute different from that of the first content on the basis of a relationship between an attribute of the first content affecting contents of a stimulus that the first content provides to the user and sleep quality indicated by the sleep data.
